(19) United States
(12) Patent Application Publication
(10) Pub. No.: US 2011/0201881 A1
(43) Pub. Date: Aug. 18, 2011

(54) LASER GUIDED ENDOSCOPIC SURGICAL TOOL

(76) Inventor: Frederick Emch, Charlottesville, VA (US)

(21) Appl. No.: 13/026,333

(22) Filed: Feb. 14, 2011

Related U.S. Application Data
(60) Provisional application No. 61/304,948, filed on Feb. 16, 2010.

(51) Int. Cl. A61B 1/01; A61B 17/32

(52) U.S. Cl. 600/108; 606/170

(57) ABSTRACT

A surgical instrument that includes a probe that is extended into a body cavity made in, for example, the wrist of a subject and a handle held by a surgeon outside the body cavity, where the surgical instrument includes both an optical system and surgical tool operable by the surgeon that passes through the probe includes a laser beam emitter to assist a surgeon in aligning the probe with, for example, the ring finger of the patient after the probe is inserted. The laser beam emitter is preferably mounted on the probe and directs a beam generally parallel to the probe which is aligned with the deployable cutting blade.

### CROSS-REFERENCE TO RELATED APPLICATIONS

This application claims priority to U.S. Provisional Patent Application 61/304,948 filed on Feb. 16, 2010, and the complete contents thereof are herein incorporated by reference.

### BACKGROUND OF THE INVENTION

1. Field of the Invention

The present invention generally relates to surgical instruments used for carpal tunnel release and similar endoscopic procedures, and is particularly directed to an improved alignment feature which allows a surgeon to more easily align the probe for proper insertion, such as, for example, alignment of the probe with the ring finger of the patient for a carpal tunnel release procedure after insertion of the probe into the patient's wrist.

2. Background Description

MicroAire Surgical Instruments has been marketing for a number of years a surgical tool based on U.S. Pat. No. 4,962,770 to Agee et al., U.S. Pat. No. 4,963,147 to Agee et al., U.S. Pat. No. 5,089,000 to Agee et al., and U.S. Pat. No. 5,306,284 to Agee et al, each of which is incorporated fully herein by reference. This surgical tool is used to inspect and manipulate selected tissue in a body cavity, and has particular application to performing safe and effective carpal tunnel release. These tools include a handle assembly, a probe member, an optical system, and a cutting system. The optical system and cutting system extend through the handle and into the probe and permit a surgical blade to be selectively deployed and retracted from a lateral opening in the top surface of the probe at its distal end.

Variations on this basic design can be found in U.S. Pat. No. 7,628,798 to Welborn which describes a locking configuration for locking the probe to the handle, and U.S. Patent Publication 2008/0195128 to Orbay and U.S. Patent 2008/0045989 to Welborn both of which describe pencil grip carpal tunnel release tools (as opposed to pistol grip designs—Arthrex Corporation commercializes a pencil grip carpal tunnel release tool), each of which is herein incorporated by reference.

The preferred use of the surgical instrument is in performing carpal tunnel release. This is accomplished by forming a short transverse incision located proximal to the carpal tunnel and the wrist flexion crease.

After longitudinal spreading dissection, to avoid injury to the sensory nerves, the incision is continued through the deep fascia of the forearm, the distal extension of which leads to the flexor retinaculum. After an incision through the finger flexor synovium, extension of the wrist will then expose the proximal opening of the carpal tunnel, thereby forming a passage to the carpal tunnel. The probe is inserted through the incision and desirably through the length of the carpal tunnel to the distal edge of the flexor retinaculum. By employing the optical system, and through manipulation of the patient's extremities, the anatomy within the carpal tunnel can be clearly visualized on a display of a video monitor connected to a video camera and lighting source associated with the optical system. The distal end of probe will desirably have gently displaced the tendons, bursa and median nerve found within the carpal tunnel to facilitate insertion of the probe. Then the lateral aperture of the probe will be positioned adjacent the medial surface of the flexor retinaculum and, desirably, the configuration of the probe upper surface (which is preferably a flat surface) will exclude the displaced tissues from the region surrounding the lateral aperture. Preferably at the appropriate location, a cutting blade will be extended to contact the distal edge of the flexor retinaculum, while the surgeon views the tissue to be divided via the display. The blade point will desirably be extended to a position sufficient to completely release the ligament. While viewing (through the lateral aperture in the probe) the intended path of the extended cutting blade, the probe is then withdrawn, thereby dividing the flexor retinaculum and releasing the carpal tunnel.

At the Rensselaer Polytechnic Institute Biomedical Engineering Senior Design Showcase, May 1, 2009, a design team working with the MicroAire Carpal Tunnel Release System developed a number of concepts for consideration. Among the ideas was the concept of using a proximity sensor configuration, comprised of a flexible tape sensor component positioned on a patient's hand and a sensor component mounted on the handpiece, together with a light spot projecting light on the flexible tape sensor component. The concept appears to provide the surgeon with some feedback on the upward or downward angle of insertion of the probe into the wrist via feedback from the proximity sensors and position of the light spot. However, this conceptual design does not appear to solve a typical problem for surgeons (it may be a solution in search of a problem), as the probe is already marked with indicia which relate to depth of insertion and the surgeon uses the optical system to judge location of the inserted probe. Rather, it would be more advantageous to know the direction that the probe is inserted, which the RPI conceptual design does not provide, and it would be advantageous to avoid having to place additional devices on the patient, such as a proximity sensor.

The surgical tool described by Agee et al. is safe and effective and well regarded in the surgical community. Aids which allow for easier alignment of the probe after insertion under the patient's skin may be perceived as advantageous to some members of the surgical community.

In an unrelated field, Pajunk GmbH of Germany has begun selling ultrasound guided nerve blocks. The product design mounts a laser pointer on a puncture cannula. The laser point projects crosslines onto a ultrasound transducer which the surgeon uses to assure that the puncture cannula is inserted into the patient at the proper upward and downward angle (45° being identified as the desired angle of insertion) so that the cannula will be precisely positioned under the ultrasound transducer. The Pajunk product is not designed for endoscopic surgery in a manner similar to the Agee, Orbay, or Welborn designs referenced above, and, similar to the RPI design project, does not provide for viewing the direction of insertion of insertable probe, rather, the downward angle of insertion is addressed.

### SUMMARY OF THE INVENTION

An alignment aid in the form of a laser beam is provided for an endoscopic surgical tool. The endoscopic surgical tool may have particular application to carpal tunnel release, as discussed in detail above in connection with Agee, Welborn, and Orbay patents and patent applications, but may also be used for other endoscopic surgical procedures. A laser beam emitter is positioned to direct a laser beam above an upper surface of said probe along a line which is generally parallel to and coplanar with said probe, and which is in alignment with a deployable cutting blade. In this way, after insertion of the probe into a subject, such as, for example an incision made in the wrist of a subject, the laser beam can be viewed by the surgeon above the skin of the patient.

This allows the surgeon to align the probe with a feature of the patient, e.g., the patient's ring finger when performing carpal tunnel release, so that it may be easier to position the probe at a desired location for cutting tissue, such as the patient's transverse ligament.

### BRIEF DESCRIPTION OF THE DRAWINGS

The foregoing and other objects, aspects and advantages will be better understood from the following detailed description of a preferred embodiment of the invention with reference to the drawings, in which:

FIG. 1 is an isometric view of a pistol grip endoscopic surgical tool with a laser diode emitter positioned in alignment with a deployable cutting blade;

FIG. 2 is an isometric view of a pencil grip endoscopic surgical tool with a laser diode emitter positioned in alignment with a deployable cutting blade;

FIG. 3 is a cut-away illustration showing the cutting blade deployed from the opening in the distal end of the probe by a tool extension system where the cutting blade is aligned with a laser beam;

FIG. 4 is exemplary embodiment showing a laser emitter being used to show a surgeon the angle of insertion of a probe in a wrist when performing carpal tunnel release surgery;

FIGS. 5a-c show exemplary isometric, end and side plan views of a diode laser emitter on a probe;

FIG. 6 shows an enlarged view of an exemplary probe which includes a diode laser emitter and a power source; and

FIG. 7 shows an isometric view of a pistol grip tool with a probe having a diode laser emitter positioned thereon.

### DETAILED DESCRIPTION

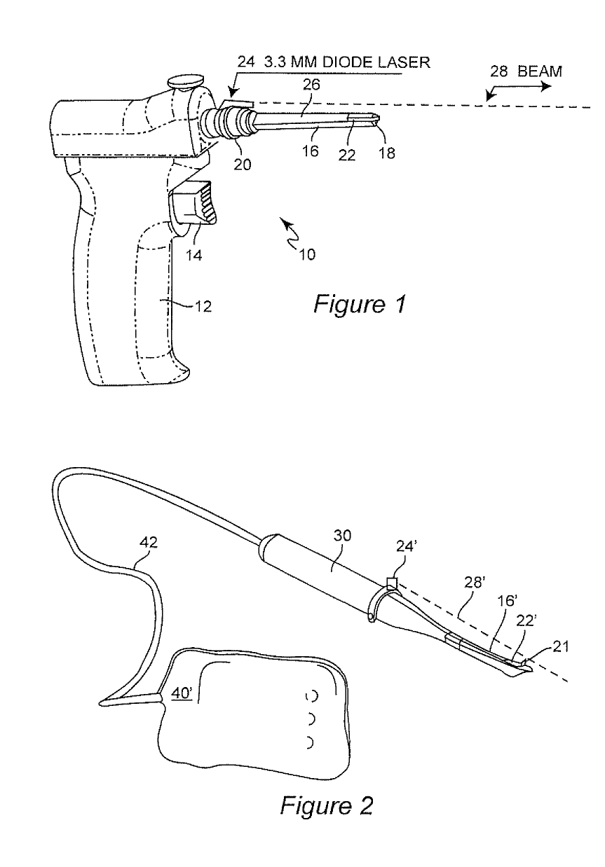

FIG. 1 shows an endoscopic surgical tool 10 configured as a carpal tunnel release tool in a manner similar to that which is described in the Agee and Welborn patents. The endoscopic surgical tool 10 includes a pistol grip handle 12 with an actuator 14. A probe 16 having a closed distal end 18 and an open proximal end 20 is connected to the pistol grip handle 12. The probe 16 has an opening 22 near its distal end 18 (i.e., it is nearer to the distal end 18 than the proximal end 20 and is preferably within one to a few millimeters). A deployable cutting blade (not shown in FIG. 1) can be deployed from opening 22 under the control of the actuator 14, and can be retracted back into the opening 22. The probe 16 is hollow and permits an optical system (e.g., one or more optical fibers, a series of lenses, etc.) to pass through such that space above the opening 22 in the probe 16 can be illuminated and imaged. A tool extension shaft also passes through the probe 16 and mechanically provides for the deployment and retraction of the cutting blade. In this way, after positioning the probe 16 in the desired location, the surgeon can view the area above the opening 22 in the probe 16 using a video system, and can determine when and where to deploy a cutting blade for separating tissue, e.g., to release the carpal tunnel ligament in carpal tunnel surgery.

The invention is focused on providing a laser beam emitter 24, such as for example a laser diode, to assist the surgeon in directing the probe 16 to the desired location after it is inserted into the patient. In one embodiment, the laser beam emitter 24 is positioned on the outer periphery of the proximal end 20 and is spaced from the top surface 26 of the probe 16 by, for example., 3-20 mm. Thus, once the probe 16 is inserted into the patient, such as for example into an incision site in the wrist of a patient, the laser beam 28 emitted from the laser beam emitter 24 can be seen by the surgeon above the skin of the patient. The laser beam 28 will be generally parallel to and coplanar with the probe 16, and will be in alignment with the cutting blade which is deployed from the opening 22 in the probe 16. This way, after, the probe 16 is inserted in the patient, the surgeon can more easily visualize the direction of the probe underneath the patient's skin by viewing the laser beam 28 above the skin. For example, in performing endoscopic carpal tunnel release, it is advantageous to have the probe 16 oriented from the incision site towards the patient's ring finger. With this invention, the surgeon merely needs to point the laser beam 28 towards the ring finger and he or she can be assured that the probe 16 is being directed to the correct location. Once the probe is in place, the surgeon can deploy the cutting blade, cut the tissue or ligament of interest, and then withdraw the probe from the patient.

While FIG. 1 shows use of the laser beam emitter 24 on a pistol grip endoscopic surgical tool, it should be understood that the inventive concept can be used with any type of endoscopic surgical tool that has a cutting blade which is deployed from an opening in a top surface of a closed ended distal probe. For example, FIG. 2 shows a laser beam emitter 24′ positioned for alignment with a probe 16′ on a pencil grip 30 tool, such as for example, the tools commercially produced by Arthrex and the tool described in the aforementioned Orbay and Welborn patent applications. The laser beam 28′ will be coplanar with and generally parallel to the probe 16′ and will be aligned with a cutting blade 21 which is deployed from an opening 22′ in the probe 16′ in the same manner as discussed above. Video images from the area above the opening 22′ can be displayed on a monitor 40 for viewing by the surgeon in order for the surgeon to decide when and where to deploy the cutting blade 21′. Data from the optical system within the probe 16′ may be provided to the monitor 40 using a cable 42 or wireless connection.

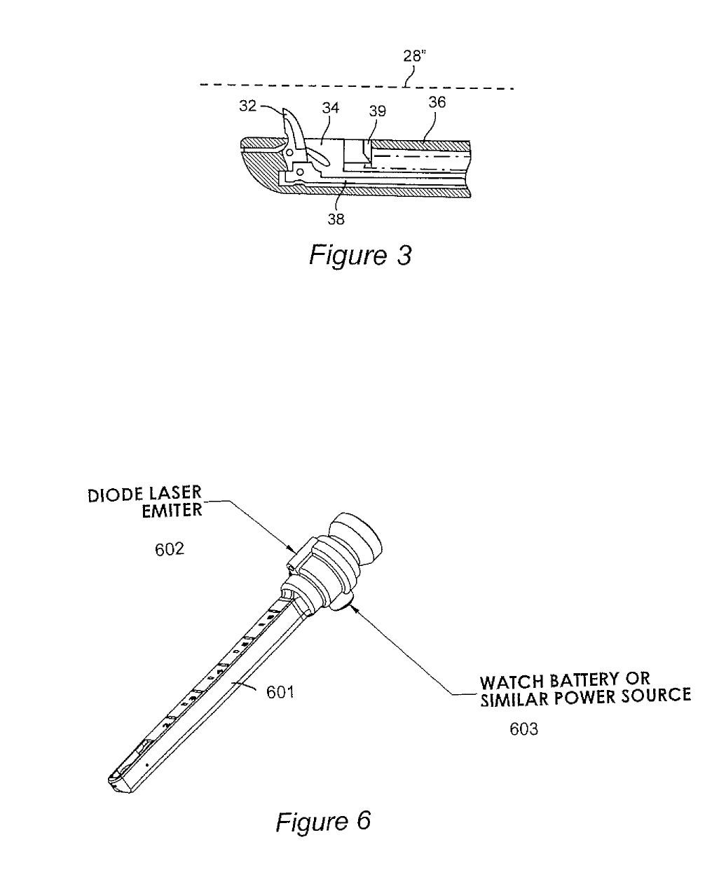

FIG. 3 shows an example of a cutting blade 32 deployed from an opening 34 in a probe 36 (it being understood that the probe 36 is the same or similar to 16 or 16′ in FIGS. 1 and 2, respectively) using a tool extension shaft 38 that passes through the probe 36 and is moveable under the control of an appropriate actuating mechanism (e.g., button, sliding mechanism, etc.). A laser beam 28″ from an emitter is aligned with the tip of the cutting blade 32, and is coplanar with and generally parallel to the probe 36. The chief requirement for the invention is that the laser beam 28″ can be viewed by the surgeon above the skin of the patient (note the separation of the laser beam 28″ above the tip of the cutting blade 32), and that it be aligned with the probe 36 and cutting blade 32. In this way, after insertion of the probe 36 into the patient, the surgeon can more easily direct the probe 36 to a desired location using the laser beam 38 as an aid. FIG. 3 also shows the end of an optical fiber 39 used for imaging and/or illuminating the area above the opening 34. As discussed in conjunction with FIG. 2, and in the incorporated references, images above the opening are used to permit the surgeon to determine when and where to deploy and retract the cutting blade 32.

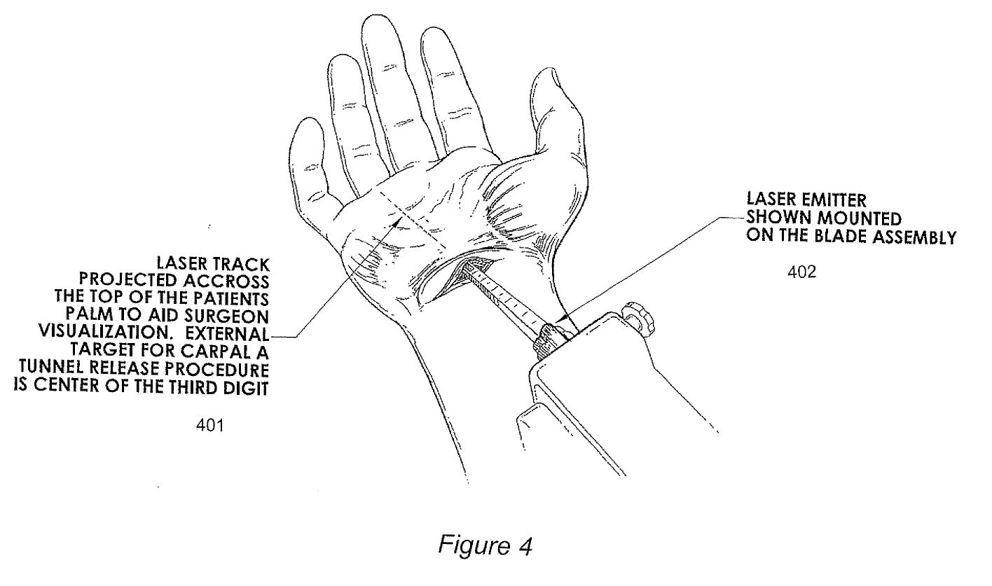

FIG. 4 illustrates a how the invention may be used in carpal tunnel release surgery. In particular, the laser track 401 from emitter 402 can be seen by the surgeon after he inserts the probe into the patient's wrist. The laser track 401 may be colored (red, green, etc.) to contrast with the patient's skin. As the emitter is projected down the same line as the probe, the surgeon is provided with an aid to visualize how the probe is moving under the skin. Specifically, in carpal tunnel release surgery, the surgeon can target the center of the third digit (i.e., the ring finger) as the direction he or she wants he probe to move towards during insertion.

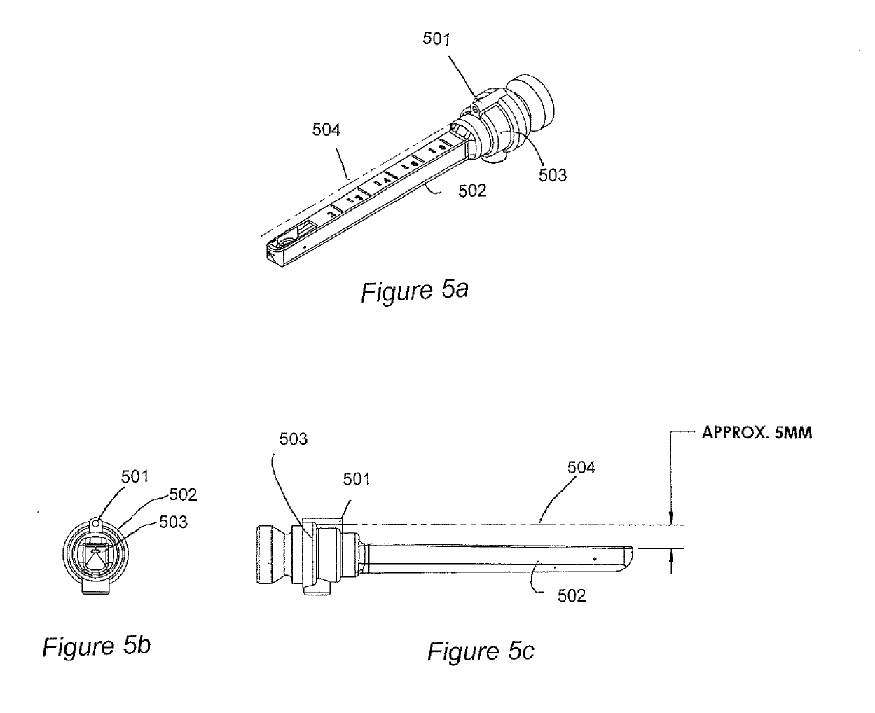

FIGS. 5a-c show exemplary isometric (5a), end (5b) and side (5c) plan views of a diode laser emitter 501 on a probe 502. The proximal end 503 of the probe 502 has a wider circumference than the probe 502. The laser emitter 501 can be molded in or connected to the proximal end 503 of the probe, and its positioning will have the laser track 504 positioned above and parallel to the probe 502. For example, if the laser track 504 is approximately 5 mm above the upper surface of the probe 502, it should be able to be viewed by the surgeon above the patient's skin (as is shown in FIG. 4). Of course, the height might vary (e.g., 3 mm to 1 cm), with the objective being that the laser track 504 is positioned high enough above the probe 502 that it can be viewed by a surgeon above the patient's skin, but not so high that the direction of orientation cannot be quickly determined by the surgeon simply by looking at laser track relative the to patient's skin (e.g., the surgeon might be looking to see the direction of the probe based on the laser track above a patient's hand in the manner illustrated in FIG. 4).

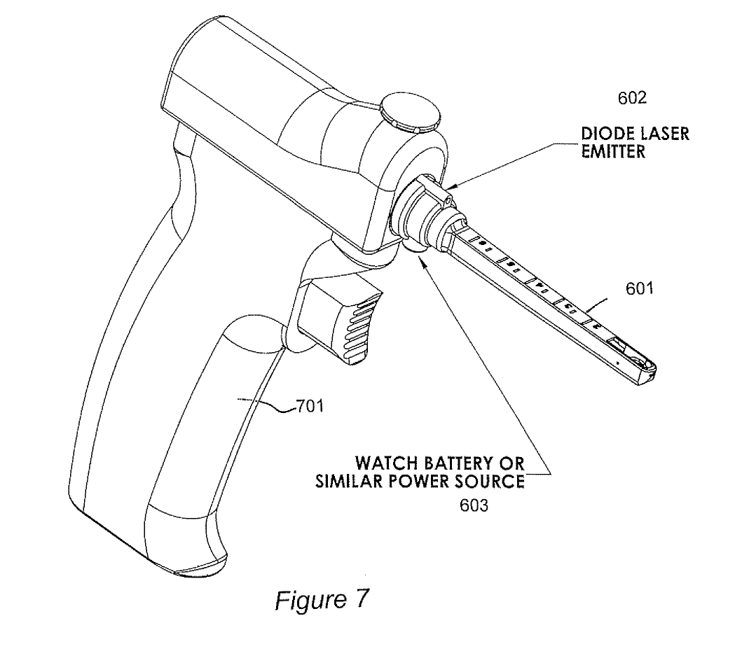

FIG. 6 shows an enlarged view of an exemplary probe 601 which includes a diode laser emitter 602 as discussed above. FIG. 6 shows a watch battery or other power source 603 might be included on the probe 601 to power the emitter 602. FIG. 7 shows the probe 601 with the emitter 602 and power source 603 connected to handpiece 701. As an alternative configuration, the probe 601 might be equipped with electrical connections at its proximal end, and power for the emitter 602 might be present in the handpiece 701.

While the invention has been described in terms of its preferred embodiments, those skilled in the art will recognize that the invention can be practiced with modification within the spirit and scope of the appended claims.

Having thus described my invention, what I claim as new and desire to secure by Letters Patent is as follows:

1. An endoscopic surgical instrument for cutting a ligament or tissue in a subject, comprising:
a probe having a closed distal end, an open proximal end, and an axial passage extending from said proximal end to said distal end through which at least an optical system and tool extension shaft extend, said probe having an upper surface with a lateral aperture positioned near said distal end through which a cutting blade can be extended and retracted under operation of said tool extension shaft;
a handle connected to said probe, said handle having an actuator which operates said tool extension shaft to extend and retract said cutting blade; and
a laser beam emitter positioned to direct a laser beam above said upper surface of said probe along a line which is generally parallel to and coplanar with said probe, and which is in alignment with said cutting blade, whereby said laser beam emitter assists a physician during endoscopic surgery to align said probe in a desired direction after insertion into a subject.

2. The surgical instrument of claim 1 wherein said handle is a pistol grip handle.

3. The surgical instrument of claim 1 wherein said handle is a pencil grip handle.

4. The surgical instrument of claim 1 wherein said laser beam emitter includes a laser diode.

5. The surgical instrument of claim 1 wherein said laser beam emitter is connected to said proximal end of said probe.

6. The surgical instrument of claim 1 wherein said laser beam emitter is positioned to emit a laser track 3 mm to 1 cm above a top surface of a said probe.

7. The surgical instrument of claim 6 wherein said laser track is colored.
